(19) United States
(12) Patent Application Publication
(10) Pub. No.: US 2014/0100178 A1
(43) Pub. Date: Apr. 10, 2014

(54) COMPOSITION AND METHODS FOR SITE-SPECIFIC DRUG DELIVERY TO TREAT MALARIA AND OTHER LIVER DISEASES

(71) Applicants: Aslam Ansari, Gaithersburg, MD (US); Sanjay Gupta, Woodbridge, VA (US)

(72) Inventors: Aslam Ansari, Gaithersburg, MD (US); Sanjay Gupta, Woodbridge, VA (US)

(21) Appl. No.: 13/645,436

(22) Filed: Oct. 4, 2012

(51) Int. Cl. A61K 47/48

(52) U.S. Cl. CPC A61K 47/48092
USPC 514/27; 536/17.4

(57) ABSTRACT

A system for selectively delivering drugs to target tissues is provided. The system includes a drug-linker-saccharide-drug conjugate (D-L-A-D1). The linker includes a functional group that is recognized and cleaved by enzyme in the target phases. The recognition segment is preferably a malaria drugs. The carrier is preferably hydrophilic, biodegradable and biocompatible particle. Any drug may be delivered using a conjugate prepared according to the invention.

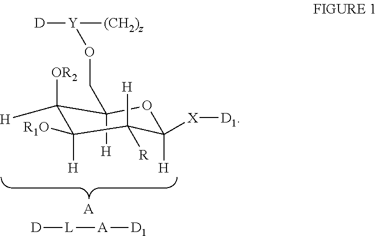

### FIELD OF THE INVENTION

The present invention provides conjugates that are capable of delivering malaria drugs for the treatment of disease, particularly in liver-phase. The liver targeting moiety consisting of chloroquine, quinine, amodiaquine, cotrifazid, doxycycline, mefloquine, proguanil, sulfadoxine-pyrimethamine, hydroxychloroquine, artmisnin derivatives, lumefatine, mefloquine, amodiaquine, sulfadoxine, pyrimethamine, atovaquone, proguanil conjugated to the galactose/galactosamine/cholesterol and their derivatives via β-linkage or through with or without another linker The covalent attachment will include antibiotics but not limited to an active metabolite or pure isomers^{25,26} (Ansari & Craig et al., synthesis, 147 (1995); chirality, 5, 88 (1993). In addition, the invention provides methods for chemical conjugations of each component into a multi-functional compound.

### SUMMARY OF THE INVENTION

The present invention provides pharmaceutical compositions and method of preparation for site-specific delivery and localization of drugs in the treatment of Malaria and other liver diseases. The compounds can be represented by the formula: D-L-A-D wherein A is an anchoring moiety; L is a linking group; D & D1 are the anti-malarial drugs. In preferred embodiments, the anchoring moiety is a galactose/galactosamine/cholesterol and their derivatives, covalently attached to the drug via glycosidic or through other reactive functional moiety and to cholesterol via amide bond. The other malarial drug D attached to the galactose through a bifunctional linker to deliver the drug in the blood. In particular, when the conjugate reaches the target, the recognition segment within the linker is thought to be cleaved by the enzyme. The active drug is thereby released from the conjugate and subsequently internalized by the cells of the target tissue

### BACKGROUND OF THE INVENTION

Malaria is the world's most serious tropical disease that currently kills more people than any other communicable disease except tuberculosis. Approximately 41% of the world's population is at risk, and each year there are an estimated 300 million to 500 million clinical cases of malaria reported Worldwide. Approximately two million deaths per year can be attributed to malaria, half of these in children¹ under five years of age and presently the number of people as infected with malarial parasites are said to have amounted to 270 millions in total. Areas in which the infection with malaria occurs are not only in the tropics, but now spreading over the temperate regions where the malarial infections were rarely found in the past due to the phenomenon of global warming,

Despite the tremendous advances made by the medical and scientific communities over the last fifty years, malaria has remained a serious endemic disease yet to be conquered by effective and adequate prophylaxis treatment or cure. One of the major factor contributing to the continued presence of malaria, during drug therapy utilizing quinine, chloroquine, amodiaquine, primaquine, and other malaria therapy is the development of resistant of malaria parasite to one or more anti-malarial drugs.

For the last 70 years, chloroquine, a synthetic drug silently served millions of lives and cured billions of debilitating episodes of malaria². This safe and inexpensive, low cost, and relative safe, led to chloroquine becoming the backbone of malaria control treatment and prevention strategies in 29 of the 42 countries in sub-Saharan Africa. (World Health Organization. 2003, Africa malaria report. World Health Organization, Geneva, Switzerland.) and to some extent all over world.

This 4-aminoquinoline compound accumulates inside the digestive vacuole of the infected red blood cell, where it believed to form complexes with toxic heme moieties and interfere with detoxification mechanism that include heme sequestration into an inert pigment called hemozoin³⁻⁵. Accumulation of haematin leads to the death of parasite. Unfortunately, use of chloroquine over several decades resulted in the development of chloroquine-resistant strains Plasmodium, particularly of P. falcifarum^{6,7} Several countries have switched their first-line drug of chloroquine to the antifolate sulfadoxine-primethamine⁸. In late 1960s combination drug sulfadoxine-pyrimethamine was introduced. But after its introduction, resistance to this drug was noted⁹. Resistance to the currently available drugs, quinine¹⁰ has also been observed, while halofantrine exhibits serious toxicity at concentrations required for treating resistant strains¹².

The inexorable spread of resistance to affordable antimalarial drugs poses one of the largest public health problems. New approaches to the use of antimalarial drugs are desperately needed in order to obtain the greatest benefit from existing antimalarial drugs, as well as to ensure that newly developed antimalarial drugs are used wisely in such a ways that could maximize their useful therapeutic life span. One such approach that has gained increasing attention worldwide was the use of antimalarial drugs in combinations called “CombinationTherapy” (CT).

The concept of combination therapy is based on the synergistic or additive potential of two or more drugs, to improve therapeutic efficacy and also delay the development of resistance to the individual components of the combination and will shorten duration of treatment (and hence increase compliance), and decrease the risk of resistant of parasites, arising through mutation during therapy.

Efficacy of combination therapy started testing (CQ+SP) and sulphadoxine-pyrimethamine (S/P), and amodiaquine (AQ) as a second-line drug to replace chloroquine¹³⁻¹⁵. Soon multi drug resistance has been reported¹⁶ from most parts of the world that alarmed to discover new antimalarial regimens urgently needed.

Artemisinins are new class of potent compounds derived from an ancient Chinese herbal remedy for malaria. These drugs exert a powerful, percussive blow, with extremely rapid killing of parasites of all ages¹⁷ of their life cycle, followed by equally rapid elimination of the drug from the body, thus avoiding the lingering sub therapeutic blood levels that creates conditions conducive to selection of resistance after treatment with slower and longer-acting drugs. In hopes of delaying the emergence of resistance to the artemisnins, the World Health Organization(WHO) recommended that they be used exclusively in combination with partner drugs that attack malaria parasites through different mechanism. The World Health Organization has recommended that artemisinin combination therapies (ACT) be first-line therapy for P. falciparum malaria worldwide^{18,19}. A large number of fixed-dose ACTs are now available containing an artemisinin component and a partner drug which has a long half-life, such as mefloquine)(ASMQ²⁰), lumefantrine (Coartem), amodiaquine (ASAQ), piperaquine (Duo-Cotecxin) and antifolates (Ariplus). There are few reports where failure of ACT is reported²³⁻²⁵.

In human, malaria disease is caused by four species of the genus Plasmodium, namely P. falciparum, P. vivax, P. ovale and P. malariae in human²⁷.

The life cycle of parasite is divided into overall three stages,
1) Mosquito
2) Liver and
3) Blood Stages

When an infected mosquito bites a human host, the sporozoites enter the blood stream and rapidly make their way to the liver, invading hepatocytes. One sporozoite can develop into 20,000 merozoites, which rupture from hepatocyte, enter the bloodstream and invade erythrocytes. This initiates a cycle of intra-erythrocytic stage. Asexual reproduction in the red cell leads to further merozoites development, leading to 10 to 20 fold increase in the number of the parasites in the blood stream every 48 hours. These asexual erythrocytic-stages of parasites are responsible for the clinical manifestations and pathology of the disease. Some erythrocytic stages differentiate into gametocytes, which are infective for mosquitoes. Fertilization occurs in the mosquito midgut and form oocysts. These oocysts rupture and releases sporozoites. During feeding, a small number of sporozoites (<100) are introduced into the salivary duct and injected into the venules of the bitten human, to initiate the cycle in the liver. The minimum time from infection with sporozoites to the first wave of merozoites that reaches the blood stream in rodent parasites (˜48 hours) while ˜15 days in human²⁸. The liver stage development of P. falciparum and P. vivax lasts 6 and 8 days, respectively^{29,30,31,32}, which is similar to many other parasite species that infect primates.

Until now all the anti malarial drugs so far developed and marketed in use are administered primarily to treat blood schizonticide, a gametocytocide and a tissue schizonticide—that is to inhibit the pathogenic, asexual blood stage parasite.

It is quite clear that life cycle of malaria disease start from liver but search for new drugs that are active against the liver stages has not received much attention.

“The hepatic stage of the life cycle is an ideal target to develop or deliver the drug to stop merozoites to invade blood circulation because this stage lasts for at least 5.5 days and is not associated with pathology. Thus full inhibition of liver stage will lead to true casual prophylaxis malaria drug.”

Primaquine is an 8-aminoquinolone that has been used for decades to prevent relapses of P. vivax and P. ovale infections (radical cure) and as a gametocidal agent to decrease the transmission of P. falciparum in malaria-endemic areas. Because primaquine has activity against both blood and tissue (liver) stages of malaria, it can eliminate P. vivax and P. falciparum infections that are developing in the liver (causal prophylaxis) and prevent symptomatic or clinical infection.

Recent randomized double blind, placebo-controlled studies have examined the efficacy of primaquine as a prophylactic agent in partially immune Kenyan children and non-immune Indonesian and Colombian men. Given at a dose of 0.5 mg/kg base per day (adult dose 30 mg base per day) for 11 to 50 weeks, primaquine had a protective efficacy of 85% to 95% against both P. falciparum and P. vivax infections. Primaquine was better tolerated than other standard chemoprophylactic regimens in persons who were not G6PD deficient.

Primaquine is generally well tolerated but may cause nausea and abdominal pain, which can be decreased by taking the drug with food. More importantly, primaquine may cause oxidant-induced hemolytic anemia with methemoglobinemia, particularly among individuals with G6PD deficiency. Primaquine is contraindicated in patients with severe G6PD deficiency. In mild variants of G6PD deficiency, primaquine has been used safely at a lower dose for radical cure to prevent P. vivax and P. ovale relapses (0.8 mg base/kg/week; adult dose 45 mg base weekly for 6 weeks); however, this reduced dose is insufficient for chemoprophylactic activity. When used at prophylactic doses (0.5 mg base/kg/day) in children and men with normal G6PD activity, mean methemoglobin rates (5.8%) were below those associated with toxicity (>10%).

Collectively, these data indicate that primaquine appears to be a safe and effective prophylactic agent in semi-immune children and non-immune adults. Theoretically, because primaquine is a causal agent, individuals should be required to take it only during periods of exposure and for 1 week after departure from the malaria-endemic area. This would avoid the requirement to complete 4 weeks of chemoprophylaxis following exposure (a common reason for non-adherence with standard regimens) and may be particularly useful for travelers with short exposures (2 to 7 days) in high-risk areas such as sub-Saharan Africa and New Guinea. Primaquine should be taken daily starting 1 day before entering a malaria-endemic area, continued while in the area and for 1 week after departure. Despite of its good oral absorption, this molecule has a very short half life (˜4 h) and needs to be administered daily. In order to achieve a therapeutic effect, primaquine was delivered via liposomal entrapment, glycoconjugate and nano-emulsion form.

During the past three decades, however, formulations that control the rate and period of drug delivery (i.e., time-release medications) and target specific areas of the body for treatment have become increasingly common. The goal of all sophisticated drug delivery systems, therefore, is to deploy medications intact to specifically targeted parts of the body through a medium that can control the therapy's administration by means of either a physiological or chemical trigger. To achieve this goal, researchers are turning to advances in the worlds of micro- and nanotechnology. Attempts have been made to develop primaquine formulation delivery by linking with lysosomotropic carriers (Trouet et al., Bulletin of World Health Organization, 59 (3): 449-458 (1981), and lipid nanoemusion formulation (Singh et al., Int. J. Pharm. 347 (2008) 136-143) to overcome both extra cellular and intracellular limitations.

The present invention provides a system for selectively delivering malaria drugs to target tissues. In preferred embodiments, the target is blood and to liver tissues. The inventive delivery system includes a -drug-linkersaccharide-drug conjugate. The linker includes a segment that is recognized and cleaved by an esterase enzyme in the blood and the other bond is cleaved in the liver target tissue. Without wishing to be bound to any particular theory, when the conjugate reaches the target tissue the recognition segment within the linker is thought to be cleaved by the enzyme. The active drug is thereby released from the conjugate and subsequently internalized by the cells of the target tissue. The physiochemical features of the carrier allow the conjugate to circulate longer in plasma by decreasing renal excretion and liver clearance. The carrier may be loaded with any number of drug molecules. In particular it is to be understood that the conjugate may include a two drug molecules each attached to the carrier via an inventive linker. It is also to be understood that any drug molecule whether a small molecule drug or a bimolecular drug (e.g., a therapeutic protein or nucleic acid) may be delivered using a conjugate prepared according to the invention.

The conjugate combination according to the present invention may conveniently be used alone or as a pharmaceutical formulation containing pharmaceutically acceptable carriers or diluents and, if desired, additional ingredients such as flavorings, binders, excipients and the like.

Pharmaceutical formulations suitable for oral administration, wherein the carrier is a solid, are in general presented as unit dose formulations such as tablets, powders, capsules, sachets and the like.

The combination of the present invention is administered preferably orally or iv, most preferably as tablets. A tablet may be made by compression or molding, optionally with one or more accessory ingredients. Compressed tablets may be prepared by compressing in a suitable machine the active compounds in a free-flowing form such as a powder or granules, optionally mixed with a binder,

Tablets containing various disintegrants such as starch, alginic acid and certain complex silicates, together with binding agents such as polyvinylpyrrolidone, sucrose, gelatin and acacia. Additionally, lubrication agents such as magnesium stearate, sodium lauryl sulfate and talc are often useful for tabletting purposes.

### EXPERIMENTAL

Galactose derivatives of anti-malarial drugs are easily prepared from the readily available starting material 1,2,3,4-Di-O-isopropylidene-α-D-Galactopyranose.

Compound 1 (1 eq) was treated with succinic anhydride (1 eq) and DMAP (0.5 eq) in dry THF for 6 h. After work-up yielded succinic acid derivatives of isopropylidene-D-galactopyranose quantitatively.

Treatment of acid derivatives of galactopyranose with DCC and amidaquine in THF yielded amidaquino-succinimidyl galactopyranose.

Hydrolysis of isopropylidene derivatives in 1NHCl/THF yielded hydroxyl derivative. It was converted into methyl-α-D-galactopyranoside by treatment with MeOH/HCl. Treatment of methyl-α-D-galactopyranoside with primaquine yielded desired product.

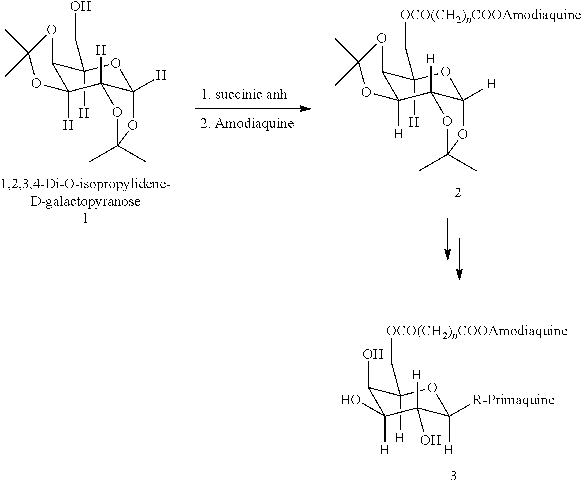

### REFERENCE

The following references are referred herein by corresponding number:
1. J. F. Trape. ‘The public health impact of chloroquine resistance in Africa. Am J Trop Med Hyg 2000, 64(suppl): 12-17
2. R. G. Ridley, Nature 415, 686 (2002).
3. D. J. Sullivan Jr., H. Matile, D. E. Goldberg, J. Biol. Chem. 273, 31103 (1998);
4. S. Pagola. P. W. Stephens, D. S. Bohle, A. D. Kosar, S. K. Madsen, Nature 404, 307 (2000);
5. H. Ginsburg, S. A. Ward, P. G. Bray, Parasitol. Today. 15, 357 (1999).
6. A. F. Slater. Pharmacol. Ther. 57, 203-235 (1993);
7. S. A. Ward. Trends. Pharmacol. Sci. 9, 241-246 (1988).
8. P. Wilairatana, D. E. Kyle, S. Looareesuwan, K. Chinwongprom, S. Amradee, N. J. White, W. M. Watkins. Ann. Trop. Med. Parasitol, 91, 125-132 (1997).
9. P. I. Trigg, A. V. Kondrachine. In Malaria. Parasite biology, pathogenesis and protection; I. W. Sherman., Ed; ASM Press: pp. 11-22 (1998)] 10. R. N. Price, C. Cassar, A. Brockman, M. Duraisingh, M. Van Vugt, N. J. Nosten, S. Krishna. Antimicr. Agents Chemother., 43, 2943 (1999)] and amadiaquinine¹¹.
11. P. G. Bray, S. R. Hawley, S. A. Ward. Mol. Pharmacol. 50, 1551 (1996).
12. F. O. Ter Kuile, G. Dolan, F. Nosten, M. D. Edstein, C. Luxemburger, L. Phaipun, T. Chongsuphajaisiddhi, H. K. Webster, N. J. White. Lancet 341, 1044 (1993).
13. East African Network for Monitoring Antimalarial Treatment (EANMAT) (2003) The efficacy of antimalarial monotherapies, sulphadoxine-pyrimethamine and amodiaquine in East Africa: Implications for sub-regional policy. Trop Med Int Health 10: 860-867).
14. olliaro P, Mussano P (2003) Amodiaquine for treating malaria (Cochrane Re-view). In: The Cochrane Library, Issue 4. Chichester, UK: John Wiley and Sons)
15. Schellenberg D, Kahigwa E, Drakeley C, Malende A, Wigayi J, et al. 0 The safety and efficacy of sulfadoxine-primethamine, amodiaquine, and their combination in the treatment of uncomplicated Plasmodium falciferum malaria. Am J trop Med Hyg 67:17-23.
16. Salah M T, Mohammed M M, Himeidan Y E, Malik E M, Elbashir M I, Adam I: Saudi Med J. 2005, 26:147-8.
17. White N J (July 1997). “Assessment of the pharmacodynamic properties of antimalarial drugs in vivo”. Antimicrob. Agents Chemother. 41 (7): 1413-22.
18. Guidelines for the Treatment of Malaria. Geneva: World Health Organization. 2006. ISBN 92-4-154694-8
19. Krudsood S, Looareesuwan S, Tangpukdee N, et al. (June 2010). “New fixed dose artesunate/mefloquine for treating multidrug resistant Plasmodium falciparum in adults—a comparative phase IIb safety and pharmacokinetic study with standard dose non-fixed artesunate plus mefloquine”. Antimicrob Agents Chemother 54 (9): 3730-7.
20. S. R. Meshnick, T. E. Taylor, S. Kamchonwongpaisan. Microbiol. Rev. 60, 301-315 (1996).
21. E. Van Geldre, A. Vergauwe, E. Van den Eeckhout. Plant Mol. Biol. 33, 199-209 (1997).
22. J. N. Cumming, P. Ploypradith, G. H. Posner. Adv. Pharmacol. 37, 253-297 (1997).
23. J. N. Cumming, D. Wang, S. B. Park, T. A. Shapiro, G. H. Posner. J. Med. Chem. 41, 952-964 (1998)
24. Adam I, A-Elbasit I E, Idris S M, Malik E M, Elbashir M I. Ann Trop Med Parasitol 2005, 99:449-55.
25. Hamour S, Melaku Y, Keus K, Wambugu J, Atkin S, Montgomery J, Ford N, Hook C, Checchi F: Trans R Soc Trop Med Hyg 2005, 99:548-54.
26. van den Broek I, Amsalu R, Balasegaram M, Hepple P, Alemu E, Hussein el B, Al-Faith M, Montgomery J, Checchi F: Malar J. 4:14 (2005).
27. Malaria: Principles and Practice of Malarialogy, Wernsdorfer, W. H., McGregor, I., Eds; Churchill-Livingstone: Edinburgh, 1988]. Of these P. falciparum is the most important as it causes almost all malaria associated deaths [Trigg, P. I., Kondrachine, A. V. In Malaria. Parasite biology, pathogenesis and protection; Sherman, I. W., Ed; ASM Press: Washington DC, 1998; pp, 11-22].
28. Lupascu, G. et al. The late primary exo-erythrocytic stages of Plasmodium malariae. Trans. Royal Soc. Trop. Med. Hyg. 61, 482-489 (1967)].
29. Boyd, M. F. & Kitchen, S. F. Observations on induced falciparum malaria. Am. J. Trop. Med. 17, 213-235 (1937).
30. Boyd, M. F. & Stratman-Thomas, W. K. Studies on benign tertian malaria. Some observations on inoculation and onset. Am. J. Hyg. 20, 488-495 (1934).
31. Bray, R. S. The exoerythrocytic phase of malaria parasites. Int. Rev. Trop. Med. 2, 41-74 (1963).
32. Fairley, N. H. Sidelights on malaria in man obtained by subinoculation experiments. Trans. Royal Soc. Trop. Med. Hyg. 40, 621-676 (1947).

What is claimed is:

1. A site specific drug delivery conjugates comprising:
a. An erythrocytic-phase drug D moiety;
b. A polyvalent linker L
c. A hepatic targeted carrier moiety A
d. A second drug active at hepatic phase D1.

2. The compound of claim 1 wherein D is one of the anti-malarial drug for erythrocytic phase, covalently attached to the linker via ester, carbamate and the linker attached to the carrier either by ester, ether or carbamate bond.

3. The compound of claim 2 wherein at least one of the drugs covalently binds to the linker is mefloquine.

4. The compound of claim 3 wherein at least D is one of the drug comprises a formula selected from the group consisting of chloroquine, quinine, amodiaquine, cotrifazid, doxycycline, mefloquine, proguanil, sulfadoxine-pyrimethamine, hydroxychloroquine, artmisnin derivatives, lumefatine, mefloquine, amodiaquine, sulfadoxine, pyrimethamine, atovaquone, proguani, sulfadiazine and sulfathiazine.

5. The drug combination of claim 1, wherein said first component of liver targeting anti-malarial drug D1, conjugated with carrier is primaquine.

6. The drug combination of claim 1, wherein said first component of liver targeting anti-malarial drug D1, conjugated with carrier is sulfathiazole or sulfathiazine.

7. The drug delivery system according to claim 6 wherein the anti-malarial conjugation is selected from the group consisting of chloroquine, quinine, amodiaquine, cotrifazid, doxycycline, mefloquine, proguanil, sulfadoxine-pyrimethamine, hydroxychloroquine, artmisnin derivatives, lumefatine, mefloquine, amodiaquine, sulfadoxine, pyrimethamine, atovaquone, proguanil.

8. The compound of claim 1 wherein the linker comprises dicarboxylic acid, hydroxyl mono and di-carboxylic acid with carbon chain ranging from C1-C30.

9. The compound of claim 6 wherein the linker comprises amino acids (natural/unnatural) where at least one carboxylic group conjugated with malarial drug D.

10. The compound of claim 9 wherein at least the linker is conjugated by amide-linked.

11. The drug delivery conjugates of claim 1 wherein the polyvalent linker includes at least one releasable linker with D.

12. The compound of claim 11 wherein the linker L further comprises one or more disulfide releasable linkers.

13. A pharmaceutical composition of claim 1 comprising a carrier, wherein said carrier is galactose or cholesterol and derivatives.

14. A pharmaceutical composition of claim 13 comprising a carrier, wherein said carrier is galactosamine.

15. A pharmaceutical composition of claim 14 comprising a carrier, wherein said Carrier is galactose and R═OH, R₁ and R₂ are H.

16. A pharmaceutical composition of claim 15 comprising a carrier, wherein said Carrier is galactosamine and R═NH₂, R₁ and R₂ are H.

17. A pharmaceutical composition of claim 14 comprising a carrier, wherein said Carrier is galactose and R═amide with C1-C30, R₁ and R₂ are alkane, alkene, alkyne, their carboxylic acid and any amine groups.

18. A pharmaceutical composition of claim 1 wherein X is amino acid, dicarboxyl, hydroxyl acid, hydroxyl aryl amine.

19. The compound of claim 18 wherein the linker X attached to the carrier by —NH, —CO, —O— and —S-bond via β-linkage

20. A pharmaceutical composition of claim 18 wherein said carrier is attached with drug directly via β-linkage.

21. A pharmaceutical composition of claim 1 wherein D and D1 are attached together as in claims 4 and 7 with all variables.

22. A pharmaceutical composition of claim 18 wherein said carrier is attached via a linker, cleavable from D1 in liver.
